(19) United States
(12) Patent Application Publication
(10) Pub. No.: US 2023/0090856 A1
(43) Pub. Date: Mar. 23, 2023

(54) Sleep Monitoring Garment and Sleep Monitoring System

(71) Applicant: FIBRET (SHANGHAI) SMART TECHNOLOGY CO., LTD., Shanghai (CN)

(72) Inventors: Zhengrong TANG, Shanghai (CN); Zhen CAI, Shanghai (CN)

(21) Appl. No.: 17/533,599

(22) Filed: Nov. 23, 2021

(30) Foreign Application Priority Data
Sep. 23, 2021 (CN) PCT/CN2021/119931

(51) Int. Cl. A61B 5/00; A61B 5/332; A61B 5/333

(52) U.S. Cl. CPC A61B 5/4806; A61B 5/6804; A61B 5/746; A61B 5/332; A61B 5/333; A61B 2562/0247; A61B 2562/0261; A61B 2562/0219; A61B 2560/0443

(57) ABSTRACT

The present disclosure provides a sleep monitoring garment and a sleep monitoring system. The sleep monitoring garment includes a wearable textile structure; a first monitoring band circumferentially extending along the wearable textile structure; a second monitoring band circumferentially extending along the wearable textile structure and with a distance to the first monitoring band; and an interface communicatively coupled with the first monitoring band and the second monitoring band.

### CROSS-REFERENCE TO RELATED APPLICATIONS

The disclosure claims the benefits of priority to PCT Application No. PCT/CN2021/119931, filed on Sep. 23, 2021, which is incorporated herein by reference in its entirety.

### TECHNICAL FIELD

The present disclosure generally relates to a wearable device, and more particularly, to a sleep monitoring garment and a sleep monitoring system.

### BACKGROUND

With the improvement of living condition, people pay more and more attention to the sleep quality. Therefore, effective sleep monitoring in daily life is in demand for understanding individual sleeping issues. Different from health monitoring for daily activities and exercise, a wearable sleep monitoring device has higher requirements for comfort.

At present, traditional sleep monitoring apparatuses are complex with wire connections, and the wires easily twist. In addition, the daily monitoring garments are often too tight and not comfortable for sleeping experience.

### SUMMARY

In some embodiments, an exemplary sleep monitoring garment includes a wearable textile structure; a first monitoring band circumferentially extending along the wearable textile structure; a second monitoring band circumferentially extending along the wearable textile structure and with a distance to the first monitoring band; and an interface communicatively coupled with the first monitoring band and the second monitoring band.

In some embodiments, an exemplary sleep monitoring system includes a sleep monitoring garment, comprising: a wearable textile structure; a first monitoring band circumferentially extending along the wearable textile structure; a second monitoring band circumferentially extending along the wearable textile structure and with a distance to the first monitoring band; and a first interface communicatively coupled with the first monitoring band and the second monitoring band; and a sleeping monitoring device, comprising: a second interface to couple with the first interface of the sleep monitoring garment; one or more sensors for detecting signals through the sleeping monitoring garment; a memory for storing the detected signals; and a processor coupled with the memory to receive processing instructions and to process the detected signals in accordance with the instructions.

Additional features and advantages of the present disclosure will be set forth in part in the following detailed description, and in part will be obvious from the description, or may be learned by practice of the present disclosure. The features and advantages of the present disclosure will be realized and attained by means of the elements and combinations particularly pointed out in the appended claims.

It is to be understood that the foregoing general description and the following detailed description are exemplary and explanatory only, and are not restrictive of the disclosed embodiments.

### BRIEF DESCRIPTION OF THE DRAWINGS

The accompanying drawings, which comprise a part of this specification, illustrate several embodiments and, together with the description, serve to explain the principles and features of the disclosed embodiments. In the drawings:

FIG. 1 is a schematic diagram of an exemplary sleep monitoring garment, according to some embodiments of the present disclosure.

FIG. 2 is an enlarged diagram of a part of the exemplary sleep monitoring garment shown in FIG. 1, according to some embodiments of the present disclosure.

FIG. 3 is another enlarged diagram of a part of the exemplary sleep monitoring garment shown in FIG. 1, according to some embodiments of the present disclosure.

FIG. 4 is another enlarged diagram of a part of the exemplary sleep monitoring garment shown in FIG. 1, according to some embodiments of the present disclosure.

FIG. 5 is a schematic diagram of the exemplary interface shown in FIG. 4 with an external device coupled thereto, according to some embodiments of the present disclosure.

FIG. 6 is a schematic diagram of an exemplary sleep monitoring system, according to some embodiments of the present disclosure.

### DETAILED DESCRIPTION

Reference will now be made in detail to exemplary embodiments, examples of which are illustrated in the accompanying drawings. The following description refers to the accompanying drawings in which the same numbers in different drawings represent the same or similar elements unless otherwise represented. The embodiments set forth in the following description of exemplary embodiments do not represent all embodiments consistent with the invention. Instead, they are merely examples of devices, systems, and methods consistent with aspects related to the invention as recited in the appended claims.

Conventional sleeping monitoring devices may be complicated, uncomfortable, and inconvenient for a user (e.g., a human being or other living being) wearing them. Embodiments of the present disclosure provide improvements over conventional sleeping monitoring devices. For example, some embodiments provide two monitoring bands attached on a garment (e.g., a pajama, a sweater, or a T-shirt etc.), which can improve a wearing experience of the user and detection precision of a sensor.

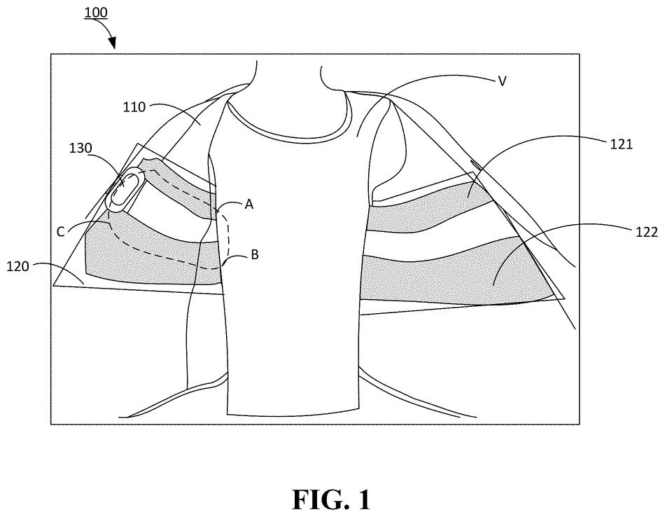

FIG. 1 is a schematic diagram of an exemplary sleep monitoring garment 100, according to some embodiments of the present disclosure. As shown in FIG. 1, the sleep monitoring garment 100 includes a wearable textile structure 110, a monitoring portion 120 and an interface 130. Although shown as a pajama, it is appreciated that wearable textile structure 110 can be in the form of other wearable textile items, such as a sweatshirt, underwear, a vest, or the like. The monitoring portion 120 is on an inner side of the garment, which is close to the body of the user, and substantially located on a portion corresponding to the upper body of the user. When lying, at least one part of the monitoring portion 120 contacts the upper body. In some embodiments, the monitoring portion 120 extends circumferentially along the garment. Therefore, the monitoring portion 120 surrounds the upper body of the user when the sleep monitoring garment 100 is worn by the user. It can be understanding that when a user is lying, there are at least several parts of the body of the user contacting a surface (e.g., floor, bed, etc.) as support points to support the body, for example, the head, the hips, the legs, the feet, the back or the arms. Due to body structure, the upper body portion always has a part that contacts the surface. For example, when a user is lying on a bed, if lying on the back, the whole back will contact the bed; if lying laterally, the side of the upper body will contact the bed; and if lying prostrate, the front of the upper body will contact the bed. Therefore, whatever the sleep posture is, when wearing the sleeping monitoring garment 100, the monitoring portion 120 around the upper body always contacts at least one part of the user (e.g., on the back, on the side, or in the front of the upper body).

Still referring to FIG. 1, the monitoring portion 120 further includes a first monitoring band 121 and a second monitoring band 122, wherein the first monitoring band 121 and the second monitoring band 122 are separated. Specifically, the first monitoring band 121 and the second monitoring band 122 extend along wearable textile structure 110 for contact with the user with a distance between them. In some embodiments, the first monitoring band 121 and the second monitoring band 122 surround, i.e., the upper body in parallel. When the sleeping user is lying in bed, the first monitoring band 121 and the second monitoring 122 both contact the user of the user.

In some embodiments, the first monitoring band 121 corresponds to a position above the heart, and the second monitoring band 122 corresponds to a position below the heart. Therefore, the monitoring can be more accurate.

The sleep monitoring garment 100 further includes an interface 130 that is communicatively coupled with the first monitoring band 121 and the second monitoring band 122, respectively. Interface 130 can be coupled to an external device, such as an electrocardiogram (ECG) recorder, a heart rate variability (HRV) monitor, a control module, a smart device, or a computer (not shown in FIG. 1), in a wired or wireless manner. When the external device is coupled to the interface 130, a circuit is formed when a user in the monitoring garment 100 is lying in bed. For example, referring to FIG. 1, when lying right laterally, the first monitoring band 121 contacts the user at point A, and the second monitoring band 122 contacts the user at point B. A circuit C is formed along the external device, the interface 130, the first monitoring band 121, the user, and the second monitoring band 122. No matter the sleep posture is, the circuit C is always connected since the first monitoring band 121 and the second monitoring band 122 always contact the user at some positions. Therefore, the external device can perform sleep monitoring continuously with the sleep monitoring garment 100.

In some embodiment, when the external device is a capacitance sensing device (e.g., a HRV monitor), the sleep monitoring garment 100 should be worn with underwear (e.g., a vest V as shown in FIG. 1) between the sleep monitoring garment 100 and the skin. The first monitoring band 121 and the second monitoring band 122 are isolated with the skin of the user by the vest insulated, and the circuit C is a capacitance path. In some embodiments, when the external device is a resistance sensing device (e.g., an ECG recorder), the sleep monitoring garment 100 should be worn next to the skin, that means, the vest (as shown in FIG. 1) should be removed. Therefore, the circuit C is a current path. There is no limit for the wearing forms of the sleep monitoring garment 100, which could be worn next to the skin or with underwear inside based on the circumstances and needs.

The sleep monitoring garment 100 described above does not necessarily need to be a tightly fitting garment, which keeps in contact with the body of the user on a fixed portion. The sleep monitoring garment 100 provided by the present disclosure can be in a loose form, and a circuit is continuously connected when the user is lying, regardless of how the body moves and whatever the posture is. Therefore, the sleep monitoring can be continuously performed during sleep and it is comfortable to wear the sleep monitoring garment 100.

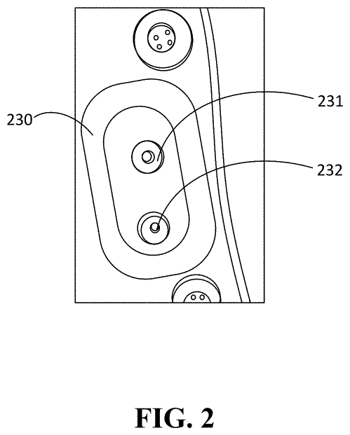

FIG. 2 is an enlarged diagram of a part of the exemplary sleep monitoring garment 100 shown in FIG. 1, according to some embodiments of the present disclosure. As shown in FIG. 2, the interface 130 can be configured as an interface 230 including a first contact 231 and a second contact 232 that are connected to the first monitoring band 121 and the second monitoring band 122, respectively (not shown in FIG. 2). An external device can be coupled to the interface 230 via the first contact 231 and the second contact 232. In some embodiments, the first contact 231 and the second contact 232 are disposed on the outside of the wearable textile structure 110, and connected with the first monitoring band 121 and the second monitoring band 122 respectively on the inner side of the wearable textile structure 110. In some embodiments, the interface 230 can be located on the front of the wearable textile structure corresponding to the chest of the upper body, and at a position between the first monitoring band 121 and the second monitoring band 122. In some embodiments, the interface 230 can be disposed on another part of the wearable textile structure 110, for example, on a cuff, on a lower hem of the wearable textile structure 110.

In some embodiments, the interface 230 can include a docking station, e.g., a recess, which can accommodate the external device and hold the external device with mechanical or magnetic force. In some embodiments, the docking station can be a hard docking station in order to better support the external device, for example a plastic docking station. In some embodiments, the docking station can be a soft docking station to provide a comfortable wearing experience, for example, the material of the docking station can be silica gel.

In some embodiments, the first contact 231 and the second contact 232 are magnetic buttons. The external device can be attached to the interface 230 with magnetic force, such that the external device can be easily detachable.

In some embodiments, the first contact 231 and the second contact 232 comprise a mechanical buckle with a locking mechanism. Therefore, the external device can be coupled to the interface 230 firmly.

In some embodiments, the interface 230 can be in other forms, for example, including a slide rail structure to fit with the external device, or having a Velcro for holding the external device.

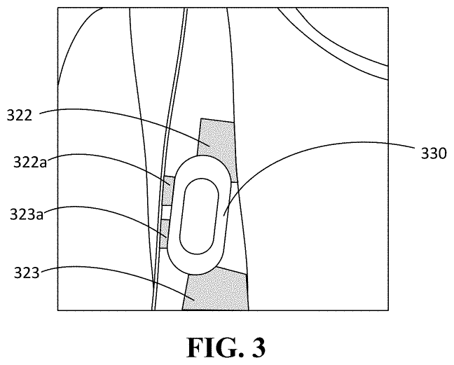

FIG. 3 is another enlarged diagram of a part of the exemplary sleep monitoring garment 100 shown in FIG. 1, according to some embodiments of the present disclosure. As shown in FIG. 3, a first monitoring band 322 includes an extension band 322a, and a second monitoring band 323 includes an extension band 323a. In this embodiment, first monitoring band 322 and second monitoring band 323 generally correspond to first monitoring band 121 and second monitoring band 122, respectively. The extension band 322a extends downwards close to the second monitoring band 323, and the extension band 323a extends upwards close to the first monitoring band 322. Therefore, the distance between the extension band 322a and the extension band 323a is smaller than the distance between the first monitoring band 322 and the second monitoring band 323. Without changing the distance between the two monitoring bands 322 and 323, the distance between the two extension bands 321a and 322a can be adjusted for communicatively coupling with the interface 330. The interface 330 is coupled to the first monitoring band 322 and the second monitoring band 323 through the extension band 322a and the extension band 323a, respectively. It is appreciated that the extension band 322a and the extension band 323a are separated. In other embodiments, the extension bands can extend to other parts of the wearable textile structure based on a design requirement, which allows the interface to be disposed on other parts of the wearable textile structure, for example, on a cuff, on a lower hem, and etc.

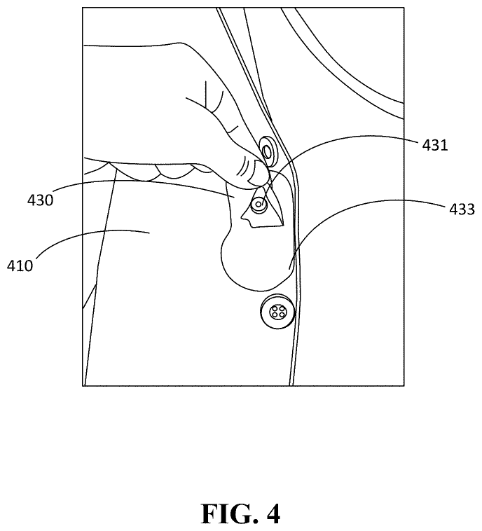

FIG. 4 is another enlarged diagram of a part of the exemplary sleep monitoring garment 100 shown in FIG. 1, according to some embodiments of the present disclosure. As shown in FIG. 4, the interface 130 can be configured as an interface 430 further including a cover 433 for covering the contacts (contact 431 for example). There is a chamber formed between the cover 433 and the wearable textile structure 410 for accommodating and protecting the external device. In some embodiments, the cover 433 has an opening. The external device can be easily put in or taken out from the opening. As shown in FIG. 4, the opening can be on the middle of the cover 433. In some embodiments, the opening is on one side of the cover, closed to the first contact or closed to the second contact. In some embodiments, the opening can be sealed by overlaps, or by Velcro, or the like. In some embodiments, the cover 433 can be made of a textile that used for the wearable textile structure. In some embodiments, the cover 433 can be transparent, for example, with a transparent plastic film, such that the external device can be seen clearly.

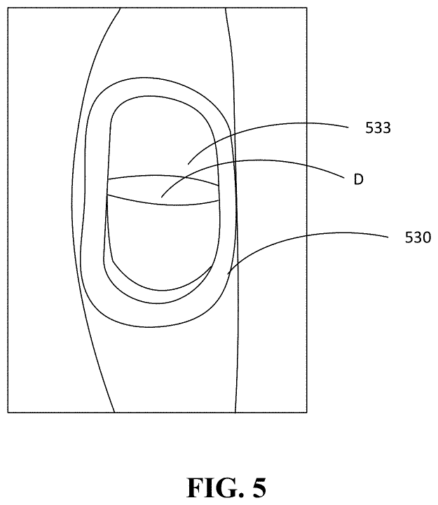

FIG. 5 is a schematic diagram of the exemplary interface 430 shown in FIG. 4 with an external device coupled thereto, according to some embodiments of the present disclosure. As shown in FIG. 5, the interface 430 can be configured as an interface 530. An external device D is coupled to the interface 530, and is covered by a cover 533. It can be appreciated that the cover 533 could be any kind of elastic textile, such that the external device D with different size can be well received in the chamber with a suitable connection with the contacts of the interface 530.

In some embodiments, the external device D can be integrated with the sleep monitoring garment 100 through the interface 530. Therefore, the connection between the external device D and the sleep monitoring garment 100 can be further ensured.

Referring back to FIG. 1, in some embodiments, the widths of each of the first monitoring band 121 and the second monitoring band 122 are between 8 cm and 15 cm, so that signal interference between them can be decreased. In some embodiments, the width of the distance between the first monitoring band 121 and the second monitoring band 122 is no greater than 12 cm, such that ECG monitoring can be more accurate. It could be understood that the widths of each of the first monitoring band 121 and the second monitoring band 122, and the width of the distance between the first monitoring band 121 and the second monitoring band 122 can be varied based on the different physical conditions of users.

In some embodiments, the first monitoring band 121 and the second monitoring band 122 can be flexible fabric electrodes, such as cotton fiber fabric electrodes, bamboo fiber fabric electrodes, polyester fabric electrodes, and any other known fabric electrodes, which can be ironed on clothing by hot pressing technology or woven into the wearable textile structure 110 by weaving.

In some embodiments, the first monitoring band 121 and the second monitoring band 122 can be interwoven or knitted into the wearable textile structure 110 with conductive yarn, serving as the electrodes. The conductive yarn can deliver electrical signals.

Furthermore, other functional yarn can be used for the monitoring bands together with the conductive yarn. The functional yarn used in the present disclosure refers to a yarn that can be used to detect various parameters of the user, such as physiological parameters, chemical parameters, local pressure, movement, gesture, or the like. The functional yarn can include, but is not limited to, pressure yarn, strain yarn, tension yarn, or the like. Conductive yarn, pressure yarn, strain yarn, or tension yarn can be used to detect electrical signals, pressure signals, strain signals, or tension signals, respectively. In some embodiments, the functional yarn can include a plurality of functional fibers twisted or interlaced together. The conductive fiber can detect and deliver electrical signals, such as physiological electrical signals of the user. The pressure fiber, strain fiber, or tension fiber can detect pressure, strain, or tension applied to the pressure fiber, strain fiber, or tension fiber, respectively. For example, an electrical resistance of the pressure fiber, strain fiber, or tension fiber can change as the pressure, strain, or tension applied to the pressure fiber, strain fiber, or tension fiber changes, respectively. Therefore, the change in electrical resistance of the pressure fiber, strain fiber, or tension fiber can be detected and used to measure a state or change in the pressure, strain, or tension applied to the pressure fiber, strain fiber, or tension fiber, respectively. The functional yarn can include a plurality of conductive fibers, pressure fibers, strain fibers, tension fibers, or a combination thereof.

In the case of using functional fibers, the interface 130 is further configured to receive the signals detected by the functional yarn and transmit the signal(s)to the external device when coupled thereto.

It could be understood that other appropriate methods could be also applied to attach the first monitoring band 121 and the second monitoring band 122 onto/into the wearable textile structure 110, which is not limit herein.

The present disclosure further provides a system including any one of the sleeping monitoring garments described above.

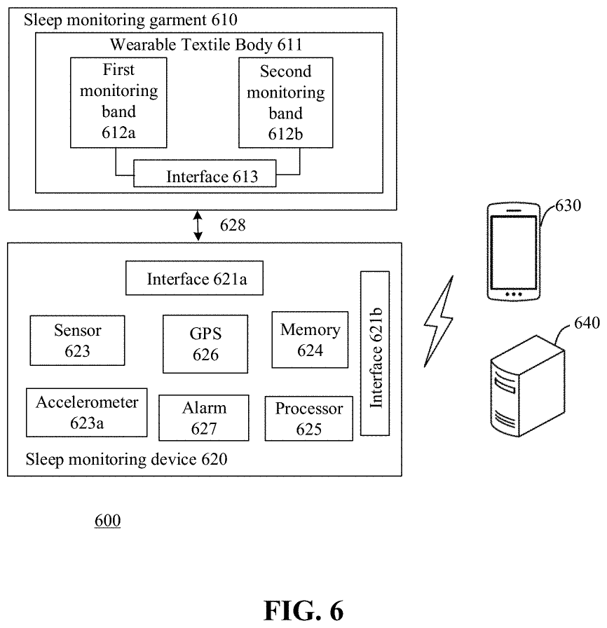

FIG. 6 is a schematic diagram of an exemplary sleep monitoring system 600 including a sleep monitoring device, according to some embodiments of the present disclosure. As shown in FIG. 6, the system 600 includes a sleep monitoring garment 610 as described above, and a sleep monitoring device 620.

The sleep monitoring garment 610 generally corresponds to the various disclosed embodiments of sleeping monitoring garment 100 and includes a wearable textile structure 611, a first monitoring band 612a, a second monitoring band 612b, and an interface 613.

The sleep monitoring device 620 includes an interface 621a, one or more sensors 623, a memory 624, and a processor 625.

Interface 621a can be wiredly or wirelessly coupled 628 to interface 613 of the sleep monitoring garment 610. For example, interface 621a can include contacts for connecting to corresponding contacts on the interface 613 of the sleep monitoring garment 610. In some embodiments, interface 613 can include a docking station, e.g., a recess, which can accommodate the sleep monitoring device 620 and hold it with mechanical or magnetic force.

The one or more sensors 623 can include, but are not limited to, a gravity sensor, an accelerometer, a magnetic field sensor, a gyroscope, or the like. In some embodiments, specifically, an accelerometer 623a is provided to assistant to determine the body movement, for example, to determine a body posture of a user (e.g., left lateral, right lateral, on the back, and etc.), or to determine a sleeping status (e.g., awake or asleep), such that a sleep stage can be determined more accurately. In some embodiments, the accelerometer 623a can be a triaxial accelerometer, a six-axis accelerometer, a nine-axis accelerometer, or any other type of accelerometer.

When the sleep monitoring device 620 is coupled to the sleep monitoring garment 610, the sensor 623 can be configured to detect ECG signals, pressure signals, tension signals, strain signals or the like.

Memory 624 can store data, signals, or instructions. For example, memory 624 can store signals detected by the sensor 623. Memory 624 can store data for processor 625 and instructions performed by processor 625. Memory 624 can be of any suitable form, including, but is not limited to, removable or nonremovable, volatile or non-volatile Read Only Memory (ROM), Random Access Memory (RAM), flash, or the like.

Processor 625 can process or pre-process signals detected by the sensor 623. The detected signals include, but are not limited to, ECG signals, pressure signals, tension signals, strain signals or the like. For example, processor 625 can pre-process detected signals to filter noise. Processor 625 can use a model or an algorithm to identify effective signals from received signals, such as heart beat signals. Processor 625 can transform received signals to data or signals indicating a situation, e.g., sleeping posture, sleeping duration, or the like, of the user. Furthermore, the processor 625 can use an algorithm to analyze sleeping quality and sleeping structure, etc.

In some embodiments, processor 625 can further encrypt data that are to be transmitted outside to protect private information of the user.

In some embodiments, the sleep monitoring device 620 can include a GPS unit 626. GPS unit 626 can provide location information of the user if the sleep monitoring device 620 is held in interface 613 of the sleep monitoring garment 610.

In some embodiments, the sleep monitoring device 620 can include an alarm unit 627. The processor 625 can further generate alert message based on a model or an algorithm with the receiving signals, and send the alert message to the alarm unit 627. Once the alarm unit 627 receives the alert message, the alarm unit 627 can start to vibrate or sound an alarm to wake the user. Furthermore, with the interface 621b which is described below, the alert message can be sent to a smart terminal 630 to warn another person (e.g., a doctor, relatives, etc.) at the same time.

In some embodiments, the sleep monitoring device 620 can include another interface 621b that can communicate with other apparatus, such as the smart terminal 630 or a server 640, in a wired or wireless manner. For example, the interface 621b can communicate with external apparatus via Universal Serial Bus (USB) or Bluetooth. The smart terminal 630 can include, but is not limited to, a cellular phone, a laptop, a tablet computer, a Personal Digital Assistant (PDA), or the like. The smart terminal 630 or server 640 can further process data from the sleep monitoring device 620. In some embodiments, the smart terminal 630 and server 640 can transmit received or further processed data to a cloud or a hospital.

Embodiments of the present disclosure provide improvements over conventional wearable devices. For example, in some embodiments, monitoring bands are ironed or interweaved on the garment for sleeping monitoring. This can provide a more comfortable wearing experience to the user. In some embodiments, the sleep monitoring garment can be coupled with a common monitoring device, such as a standard ECG recorder. In addition, the wearable device according to some embodiments of the present disclosure can provide real-time monitoring with high performance.

The embodiments may further be described using the following clauses:

1. A sleep monitoring garment, comprising:
a wearable textile structure;
a first monitoring band circumferentially extending along the wearable textile structure;
a second monitoring band circumferentially extending along the wearable textile structure and with a distance to the first monitoring band; and
an interface communicatively coupled with the first monitoring band and the second monitoring band.

2. The sleep monitoring garment of clause 1, wherein the first monitoring band and the second monitoring band corresponds to a position around an upper body of a user.

3. The sleep monitoring garment of clause 2, wherein the first monitoring band corresponds to a position above the heart of the user, and the second monitoring band corresponds to a position below the heart of the user.

4. The sleep monitoring garment of any one of clauses 1 to 3, wherein the first monitoring band and the second monitoring band are flexible fabric electrodes.

5. The sleep monitoring garment of clause 4, wherein the first monitoring band and the second monitoring band are ironed on the wearable textile structure.

6. The sleep monitoring garment of clause 4, wherein the first monitoring band and the second monitoring band are woven on the wearable textile structure.

7. The sleep monitoring garment of any one of clauses 1 to 3, wherein the first monitoring band and the second monitoring band comprise conductive fibers interwoven into the wearable textile structure.

8. The sleep monitoring garment of clause 7, wherein the first monitoring band and the second monitoring band further comprise a plurality of pressure fibers, strain fibers, or tension fibers.

9. The sleep monitoring garment of any one of clauses 1 to 8, wherein the first monitoring band comprises a first extension band and the second monitoring band comprises a second extension band, and the interface is connected with the first extension band and the second extension band.

10. The sleep monitoring garment of any one of clauses 1 to 9, wherein the interface further comprises:
a first contact coupled with the first monitoring band; and
a second contact coupled with the second monitoring band.

11. The sleep monitoring garment of clause 10, wherein the first contact and the second contact are magnetic buttons.

12. The sleep monitoring garment of any one of clauses 1 to 11, wherein the interface further comprises:

a cover for covering the interface, and a chamber formed between the cover and the wearable textile structure.

13. The sleep monitoring garment of clause 12, wherein the cover comprises an opening for access to the chamber.

14. A sleep monitoring system, comprising:
a sleep monitoring garment, comprising:
   a wearable textile structure;
   a first monitoring band circumferentially extending along the wearable textile structure;
   a second monitoring band circumferentially extending along the wearable textile structure and with a distance to the first monitoring band; and
   a first interface communicatively coupled with the first monitoring band and the second monitoring band; and
a sleeping monitoring device, comprising:
   a second interface to couple with the first interface of the sleep monitoring garment;
   one or more sensors for detecting signals through the sleeping monitoring garment;
   a memory for storing the detected signals; and
   a processor coupled with the memory to receive processing instructions and to process the detected signals in accordance with the instructions.

15. The sleep monitoring system of clause 14, wherein the sleep monitoring device further comprises an accelerometer for detecting a body movement.

16. The sleep monitoring system of clause 14 or 15, wherein the sleep monitoring device is removable from the sleep monitoring garment.

17. The sleep monitoring system of clause 14 or 15, wherein the sleep monitoring device is integrated with the sleep monitoring garment.

18. The sleep monitoring system of clause m 14, wherein the sleep monitoring device further comprises a third interface for communicating with an external apparatus.

19. The sleep monitoring system of clause 14, wherein the sleep monitoring device further comprises an alarm unit for waking a user in response to an alert message.

20. The sleep monitoring system of clause 14, wherein the sleep monitoring device is an electrocardiogram (ECG) recorder.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive.

The features and advantages of the present disclosure are apparent from the detailed specification, and thus, it is intended that the appended claims cover all systems and methods falling within the true spirit and scope of the present disclosure. As used herein, the indefinite articles “a” and “an” mean “one or more.” Further, since numerous modifications and variations will readily occur from studying the present disclosure, it is not desired to limit the present disclosure to the exact reconstruction and operation illustrated and described, and accordingly, all suitable modifications and equivalents may be resorted to, falling within the scope of the present disclosure.

As used herein, unless specifically stated otherwise, the term “or” encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A sleep monitoring garment, comprising:
a wearable textile structure;
a first monitoring band circumferentially extending along the wearable textile structure;
a second monitoring band circumferentially extending along the wearable textile structure and with a distance to the first monitoring band; and
an interface communicatively coupled with the first monitoring band and the second monitoring band.

2. The sleep monitoring garment of claim 1, wherein the first monitoring band and the second monitoring band corresponds to a position around an upper body of a user.

3. The sleep monitoring garment of claim 2, wherein the first monitoring band corresponds to a position above the heart of the user, and the second monitoring band corresponds to a position below the heart of the user.

4. The sleep monitoring garment of claim 1, wherein the first monitoring band and the second monitoring band are flexible fabric electrodes.

5. The sleep monitoring garment of claim 4, wherein the first monitoring band and the second monitoring band are ironed on the wearable textile structure.

6. The sleep monitoring garment of claim 4, wherein the first monitoring band and the second monitoring band are woven on the wearable textile structure.

7. The sleep monitoring garment of claim 1, wherein the first monitoring band and the second monitoring band comprise conductive fibers interwoven into the wearable textile structure.

8. The sleep monitoring garment of claim 7, wherein the first monitoring band and the second monitoring band further comprise a plurality of pressure fibers, strain fibers, or tension fibers.

9. The sleep monitoring garment of claim 1, wherein the first monitoring band comprises a first extension band and the second monitoring band comprises a second extension band, and the interface is connected with the first extension band and the second extension band.

10. The sleep monitoring garment of claim 1, wherein the interface further comprises:
a first contact coupled with the first monitoring band; and
a second contact coupled with the second monitoring band.

11. The sleep monitoring garment of claim 10, wherein the first contact and the second contact are magnetic buttons.

12. The sleep monitoring garment of claim 1, wherein the interface further comprises:
a cover for covering the interface, and a chamber formed between the cover and the wearable textile structure.

13. The sleep monitoring garment of claim 12, wherein the cover comprises an opening for access to the chamber.

14. A sleep monitoring system, comprising:
a sleep monitoring garment, comprising:
a wearable textile structure;
a first monitoring band circumferentially extending along the wearable textile structure;
a second monitoring band circumferentially extending along the wearable textile structure and with a distance to the first monitoring band; and
a first interface communicatively coupled with the first monitoring band and the second monitoring band; and
a sleeping monitoring device, comprising:
a second interface to couple with the first interface of the sleep monitoring garment;
one or more sensors for detecting signals through the sleeping monitoring garment;
a memory for storing the detected signals; and
a processor coupled with the memory to receive processing instructions and to process the detected signals in accordance with the instructions.

15. The sleep monitoring system of claim 14, wherein the sleep monitoring device further comprises an accelerometer for detecting a body movement.

16. The sleep monitoring system of claim 14, wherein the sleep monitoring device is removable from the sleep monitoring garment.

17. The sleep monitoring system of claim 14, wherein the sleep monitoring device is integrated with the sleep monitoring garment.

18. The sleep monitoring system of claim 14, wherein the sleep monitoring device further comprises a third interface for communicating with an external apparatus.

19. The sleep monitoring system of claim 14, wherein the sleep monitoring device further comprises an alarm unit for waking a user in response to an alert message.

20. The sleep monitoring system of claim 14, wherein the sleep monitoring device is an electrocardiogram (ECG) recorder.
